(19) United States
(12) Patent Application Publication
(10) Pub. No.: US 2012/0289759 A1
(43) Pub. Date: Nov. 15, 2012

(54) INTERLOCKING VACUUM CUSHION

(76) Inventor: MICHAEL LYNCH, York, PA (US)

(21) Appl. No.: 13/465,156

(22) Filed: May 7, 2012

Related U.S. Application Data
(60) Provisional application No. 61/485,722, filed on May 13, 2011.

(51) Int. Cl. A61F 5/37; A61N 5/00

(52) U.S. Cl. 600/1; 128/870

(57) ABSTRACT

A method of positioning, holding, and immobilization of a patient on a radiation treatment table or patient support surface. The positioning device is an improved vacuum cushion for the immobilization of a patient's full body on a support surface for some type of therapy to be accurately delivered and repeated. The patient's body is to be positioned in a manner for treatment that can be reproduced during consecutive days of radiotherapy. The upper and lower body cushions both include an outer casing with a valve that is connectable to a vacuum source. The casings contain small spheres. The immobilization device is comprised of upper and lower body cushions, both non-porous, to be evacuated of air. The cushions are adjoined by interlocking frame members fixed at proximal ends of each cushion. The device can be secured on the support surface at predetermined points using commercially available indexing bars during the procedure.

### CROSS-REFERENCE TO RELATED APPLICATIONS

This application is claiming the benefit, under 35 U.S.C. §119(e), of the provision application filed on May 13, 2011 under 35 U.S.C. §111(b), which was granted Ser. No. 61/485,722. This provisional application is hereby incorporated by reference in its entirety.

### STATEMENT REGARDING FEDERALLY SPONSORED RESEARCH OR DEVELOPMENT

Not Applicable

### BACKGROUND OF THE INVENTION

The present invention is in the technical field of Radiation Therapy. More particularly, the present invention relates to positioning, holding, and immobilization of a patient on a radiation treatment table or patient support surface.

The field of radiation therapy is currently using a technique known as stereotactic body radiation therapy (SBRT). SBRT can be used to precisely target a tumor anywhere in the body. Stereotactic pertains to the accurate localization of tumor volume in relationship to the body. This technique requires fewer fractions and higher doses per fraction. The advantages include a shorter treatment period, less time spent traveling to the treatment center, fewer side effects, and preferable tumor response. This form of treatment has been made possible by the vast improvement of imaging capabilities found on linear accelerators. The ability to visualize tumors and surrounding structures enable a precise treatment with pinpoint accuracy.

Patient positioning systems used during radiation therapy and diagnostic imaging ensure accurate patient position, immobilization, and reproducibility. Typically, a moldable vacuum cushion or stereotactic frame is used. Such devices are disposed on top of various support surfaces or treatment tables associated with radiation treatment or imaging equipment. Such commercially available support surfaces have equidistant indents successively along their longitudinal edges. Commonly, these indents are used to fixate positioning and or immobilization devices in predetermined positions in relationship to the support surface. This fixation between device and support surface is made by a releasable elongated bar with two pins projecting upward to be received in corresponding holes of the positioning and or immobilization device. Indexing the patient and devices to the same index point on the support surface increases treatment accuracy and consistency.

The cushions generally used are made of a flexible urethane or nylon casing approximately 0.008-0.012 inch-thick, containing small spheres normally made of polystyrene, approximately 0.070 inch in diameter. The cushions may have a horizontally fixed frame to accept the pins on an index bar. In molding the patient positioning cushion, the cushion is secured on top of the support surface in its inflated state making it soft and moldable. The patient lies supine on top of the cushion. A partial vacuum is created by evacuating air from the cushion through a valve using a vacuum compressor. The cushion is molded around the patient's body contours and the remaining air is evacuated through the valve. The cushion is a rigid mold that will hold a particular patient in a desired and reproducible position for precise radiation treatments. Once the patient completes their course of treatment the cushion is inflated and cleaned to be used for the next patient.

SBRT requires a high level of accuracy and patient positioning is vital to ensure that tumor volume is targeted and exposure to healthy tissue is minimized. To ensure this level of reproducibility in patient positioning it is imperative that the vacuum mold retain its shape and maintain close repositioning tolerances throughout the course of treatment. Although, conventional full body cushions perform fairly well, they can be quite difficult for patients to enter and exit. To sufficiently immobilize a patient the cushion is molded around the sides of the body creating a deep concave shape. The sidewalls of the cushion can become quite tall along the torso region requiring the patient to use a step ladder and support one's body weight on the walls of the cushion to enter and exit the molded cushion. Repeated entering and exiting of the patient from the mold can quickly distort its shape and isocenter markings. The difficult and sometimes unsafe act of gaining access to the cushion can result in undesirable patient positioning.

Prior art examples include U.S. Pat. No. 5,832,550 ('550) by Hauger et al. entitled “Moldable Vacuum Cushion” and US Patent Application Publication US 2011/0271451 ('451) by Huttner et al. entitled “Vacuum Patient Positioning Cushion With Integrated Rigid or Semi-Rigid Positioning Element.” The '550 patent discloses a moldable vacuum cushion for positioning a patient during radiation therapy treatment including an indexing bar with indexing pins to allow the attached cushion to be quickly, easily, accurately and repeatedly indexed on a base plate or treatment table. The indexing bar may be releasably mounted on affixed to the cushion or may be directly mounted on the cushion. The '451 Patent Application Publication discloses a vacuum patient positioning cushion that includes an integrated rigid or semi rigid positioning element located within a gas impermeable outer cover, making it possible to easily and correctly position a patient for a radiation therapy or other procedure without the need for or use of external positioning devices. The gas-impermeable outer covering defines an air tight enclosure having valve stem and closeable vacuum control valve in selective fluid communication with the enclosure. The enclosure houses a filler material and a rigid or semi-rigid positioning element. A method of positioning a patient using the vacuum patient positioning cushion is also provided.

Accordingly, the primary objective of the present invention addresses the need for an improved moldable cushion system that provides immobilization and reproducible patient positioning during SBRT, radiation therapy treatments, and other medical imaging procedures.

Another objective of the present invention is the improvement of reproducing close tolerances in patient positioning throughout repeated treatments and medical imaging procedures.

Another objective of the present invention is the optimization of patient safety and comfort.

Another objective of the present invention is the ease of use and adaptability to various positioning/immobilization methods suiting the user's therapy equipment and treatment plan.

Another objective of the present invention is that the size, shape and fill volume of the upper body cushion and the lower body cushion may be configured to suit the user's needs.

These objectives, along with others will become evident in the following description of the present invention.

### SUMMARY OF THE INVENTION

The present invention is an improved vacuum cushion for the immobilization of a patient's full body on a support surface for some type of therapy to be accurately delivered and repeated. The patient's body is to be positioned in a desirable manner for treatment that can be reproduced during consecutive days of radiotherapy. The upper and lower cushions both include an outer casing with a valve that is connectable to a vacuum source. The casings contain small spheres. The immobilization device is comprised of upper body and lower body cushions, both non-porous, to be evacuated of air. The cushions are adjoined by interlocking frame members fixed at proximal ends of each cushion. In one embodiment, fixed to the outer casing of the upper and lower cushions are cushion frame members that can be connected to indexing bars. The device can be secured on the support surface at predetermined points using commercially available indexing bars during the procedure. Alternatively, the device may be used without indexing bars and instead molded on top of various patient positioning aids, e.g., stereotactic frames, wedges, wing boards, headrests, etc., that are indexed to the support surface. Upon evacuation of air from both upper and lower cushions, the patient's body is held tightly in place. The device retains the patient's contour and position. The upper body and lower body cushions can be quickly and easily separated and reattached, using the interlocking mechanism. Separating the lower body cushion from the upper body cushion increases the patient's ability to enter and exit the cushion thus preserving the mold's shape, rigidity, patient position, and body contour.

### BRIEF DESCRIPTION OF THE DRAWINGS

FIG. 1 illustrates a perspective top view of one embodiment of a patient positioned in the attached cushions in the molded and evacuated state;

FIG. 2 a perspective top view of the cushions attached;

FIG. 3 illustrates a perspective bottom view of the cushions attached;

FIG. 4 illustrates a perspective top view of the cushions separated;

FIG. 5 illustrates a partial top view of the separated upper body and lower body cushions' frame members;

FIG. 6 illustrates a partial top view of the attached upper body and lower body cushions' frame members;

FIG. 7 illustrates a top view of the upper body and lower body cushions' interlocking frame members;

FIG. 8 illustrates a front view of the upper body cushion's interlocking frame member;

FIG. 9 illustrates a front view of the lower body cushion's interlocking frame member;

FIG. 10 illustrates a Cross-sectional view of front upper body cushion's interlocking frame member viewed along line 10-10 in FIG. 5;

FIG. 11 illustrates an enlarged cross-sectional view of FIG. 10 in unlocked position.

FIG. 12 illustrates an enlarged cross-sectional view of FIG. 10 in locked position;

FIG. 13 illustrates a top cross-sectional view of the upper body and lower body cushions' interlocking frame members separated;

FIG. 14 illustrates a top cross-sectional view of the upper body and lower body cushions' interlocking frame members attached;

FIG. 15 illustrates a top cross-sectional view of the upper body and lower body cushions' interlocking frame members attached and locked;

FIG. 16 illustrates an enlarged view of FIG. 13;

FIG. 17 illustrates a right cross-sectional view of one embodiment of the upper body and lower body cushions attached and positioned above a treatment table with indexing bars;

FIG. 18 illustrates a bottom view of an upper body cushion and a lower body cushion interlocked and having alternative cushion position frame members.

FIG. 19 illustrates a bottom view a lower body cushion with cushion position member in an unlocked mode.

FIG. 20 illustrates a view of a cushion with an illustration of the alternative cushion position frame member.

These and other features, aspects, and advantages of the present invention will become better understood with reference to the following description and appended claims.

### DETAILED DESCRIPTION OF THE INVENTION

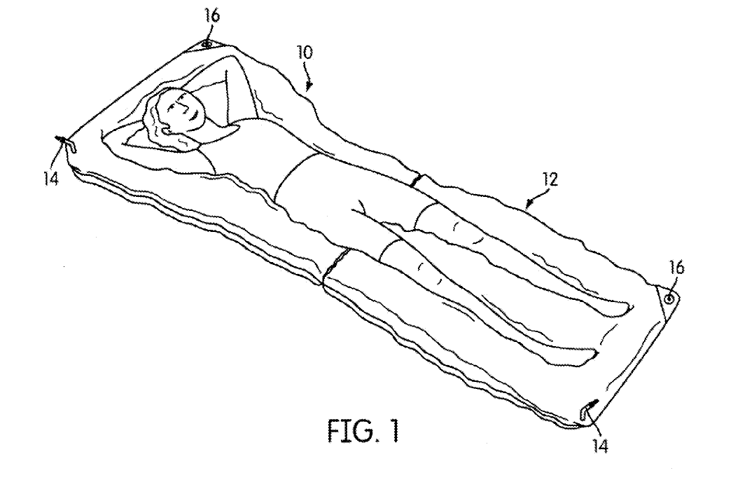

FIG. 1 illustrates a pair of moldable vacuum cushions 10 and 12. With reference to the drawings, the numerals 10 and 12 designate a moldable vacuum cushion. Such cushions are utilized to position or immobilize a patient for radiation therapy treatments or medical imaging. Typically a cushion 10 and 12 has an outer casing made from a flexible, gas impermeable material such as nylon reinforced urethane. The cushions are filled with a yieldable substance such as polystyrene spheres. A valve 14 is use to inflate and deflate the cushion. A ring 16 is used to hang the cushions during storage.

Referring now to the interlocking vacuum cushions 10 and 12 in more detail, FIG. 1 illustrates a patient lying in a possible treatment position encased by a cushion 10 and 12. The upper body and lower body cushions 10 and 12 (as related to a patient) are interlocked approximately inferior to the buttocks. The upper body cushion 10 and lower body cushion 12 are shown in their evacuated state molded around the patient's body contours. In this state the cushions create a rigid mold of the body. The body cushions 10 and 12 are sufficiently long and wide* enough to encompass a patient's body. The size, shape and fill volume of the upper body cushion and the lower body cushion may be configured to suit the user's needs. The cushions are ideally molded upward around the body to create a hollowed mold of the body once evacuated of air. The ability to separate the lower body cushion 12 from the upper body cushion 10 is highly advantageous. With the lower body cushion 12 removed, the patient is able to enter and exit the upper body cushion 10 without supporting his/her body weight on the sidewall of the mold. When the patient is in position the lower body cushion 12 can be attached.

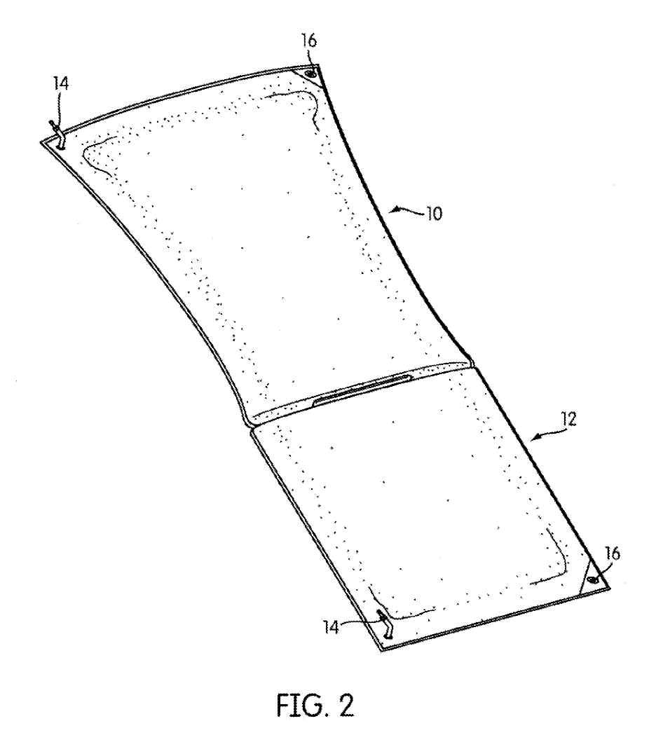

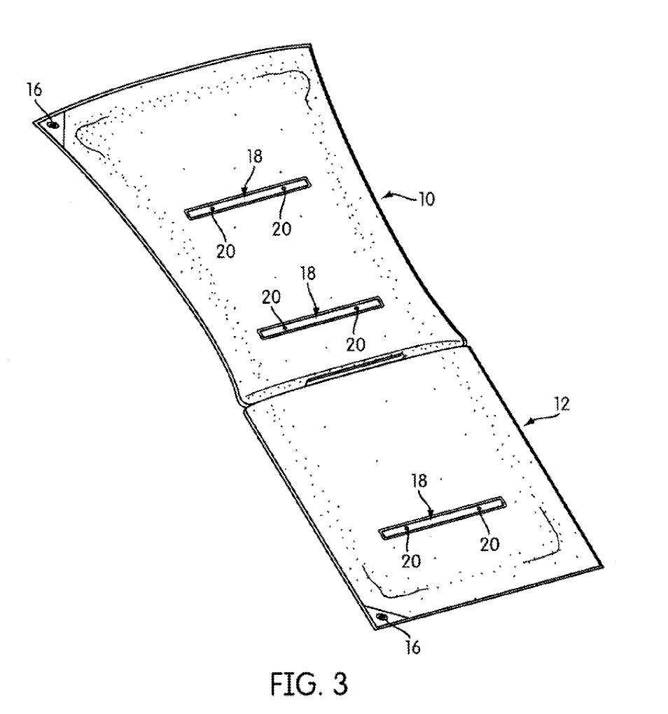

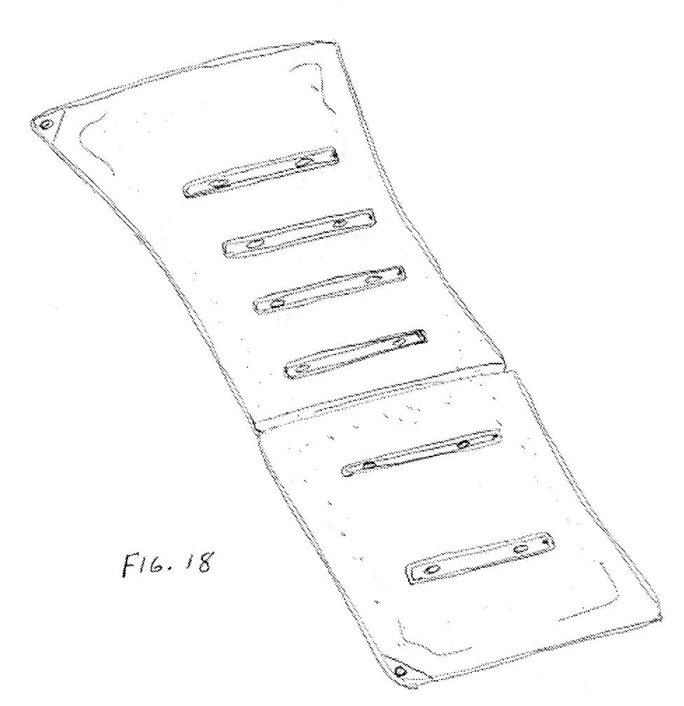

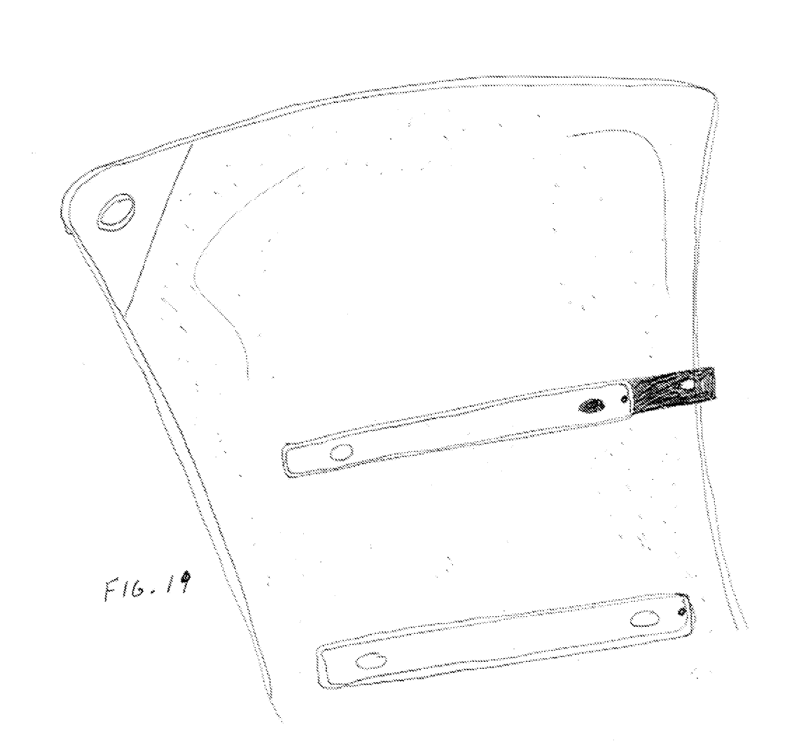

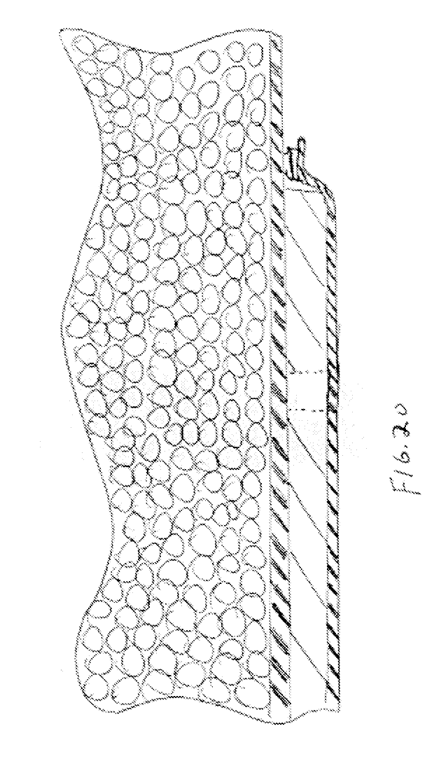

FIGS. 2 and 3 illustrate the upper cushion 10 and lower cushion 12 attached in their inflated state. As best seen in FIGS. 3 and 17, an embodiment has cushion position frame members 18 sandwiched between two layers of the casing material 11. Alternatively as illustrated in FIGS. 18, 19 AND 20 the cushion frame members may be contained within a sleeve created by the casing material 11. Either end of the sleeve may have a flap. The flap can be secured with Velcro or a snap. One advantage of the alternative cushion positioning members is that cushion position frame members can be added or removed as needed. Each cushion position frame member 18 contains holes 20 which can accept the pins of an indexing bar 48 as shown in FIG. 17. Commercially available indexing bars 48 are commonly used to secure positioning devices to the treatment table 50.

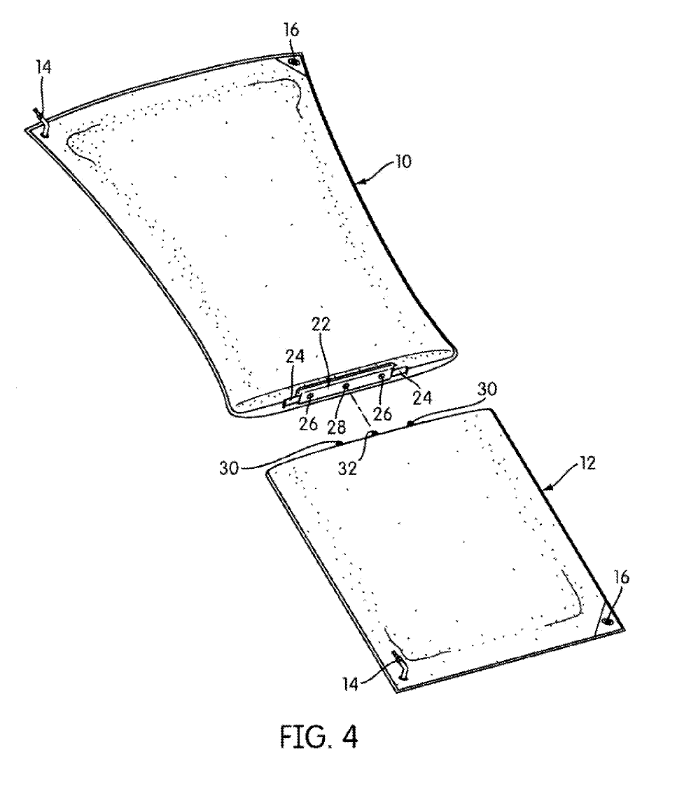

Referring now to FIG. 4, the upper body cushion 10 and lower body cushion 12 are illustrated in separate positions. Located at the proximal end of the upper body cushion 10 is shown an interlocking frame member 22 with holes 26 and 28 and slide latches 24. The outer holes 26 accept corresponding outer pins 30 and the center hole 28 accepts a corresponding center pin 32.

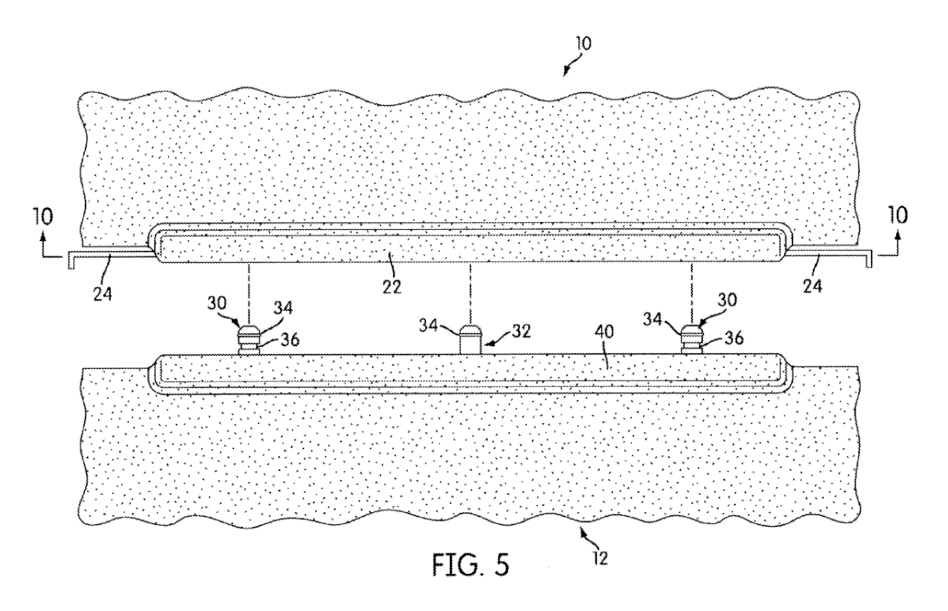

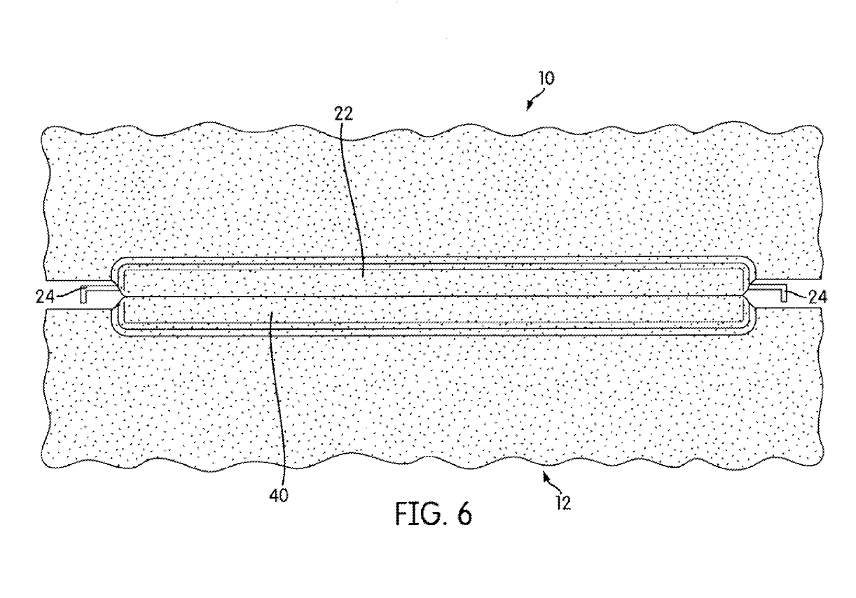

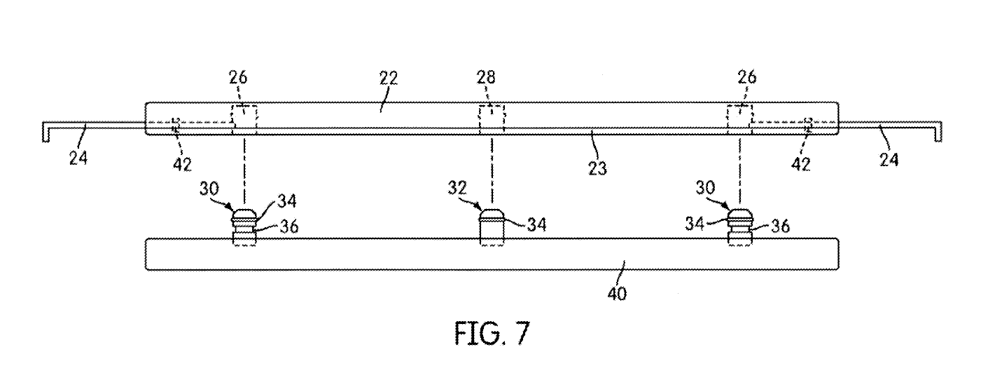

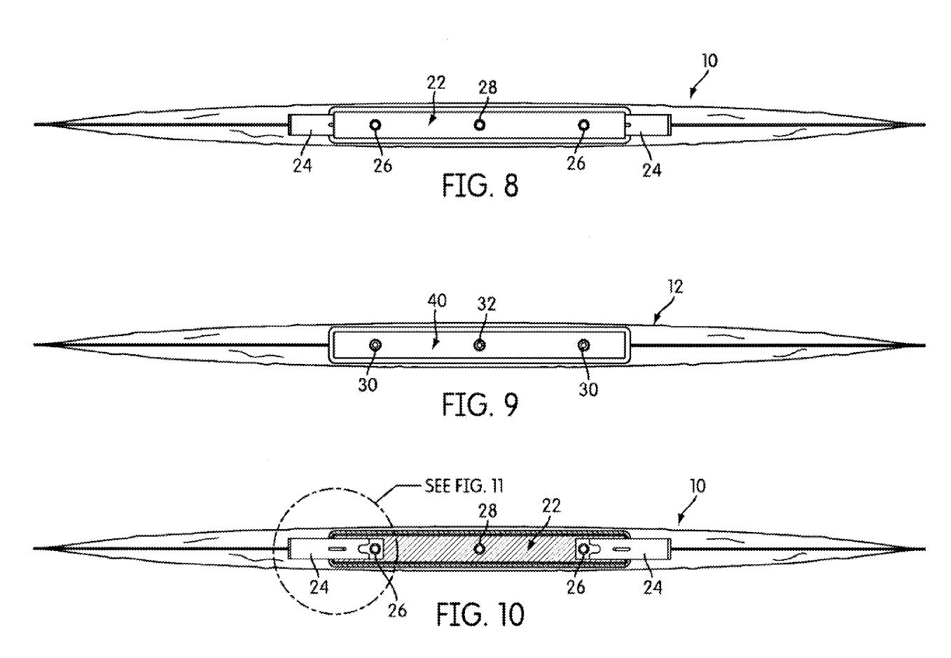

FIGS. 5 and 6 illustrate a pair of interlocking frame members 22 and 40 located at proximal ends of each body cushions 10 and 12. The outer pins 30 have a groove 36 and rubber O-ring 34. The center pin 32 has a rubber O-ring 34. Found at each end of the frame member 22 are slide latches 24. The location of the holes on the upper body cushion and the pins on the lower body cushion as shown in FIGS. 4-10 may be reversed by a manufacturer if desired.

As illustrated in FIGS. 7, 8, 9 and 10, the two outer pins 30 and a center pin 32 are counter sunk into the interlocking frame member 40. For added strength they are further secured with an epoxy or adhesive. The outer pins 30 and the center pin 32 of the lower frame member 40 have corresponding holes 26 and 28 located in the upper frame member 22. Located within the upper frame member 22 are two slide latches 24. The slide latches 24 are encased by an outer plate 23. Dowel pins 42 are counter sunk into the frame member 22 and outer plate 23. The outer plate 23 is mated to the frame member 22 using an epoxy or adhesive.

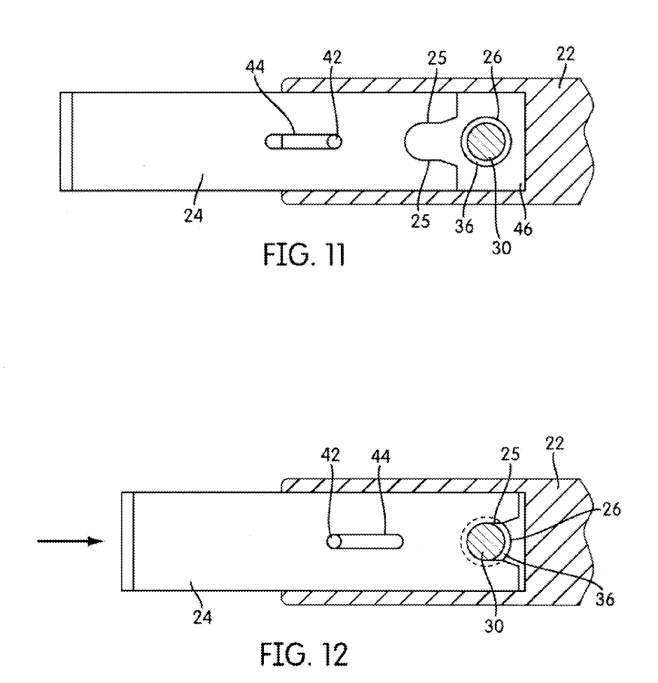

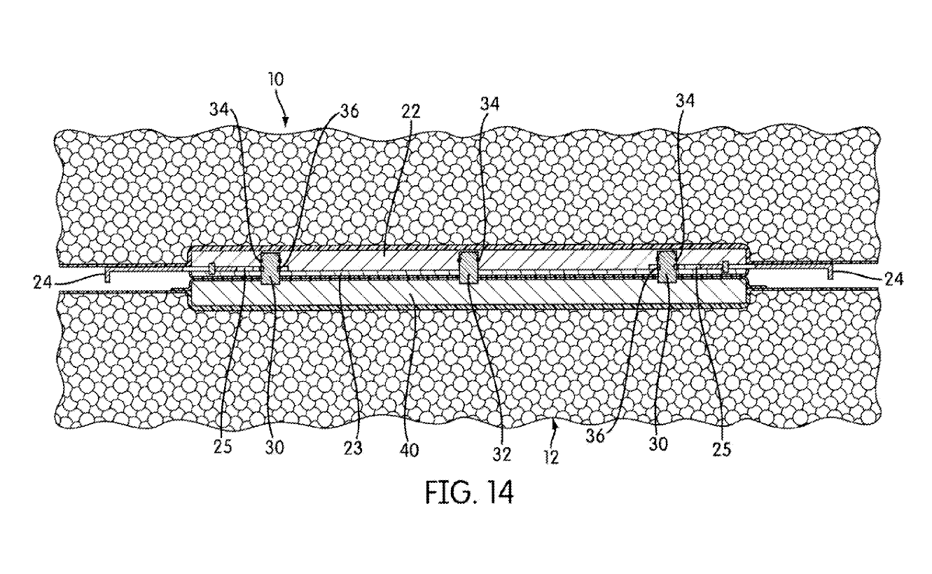

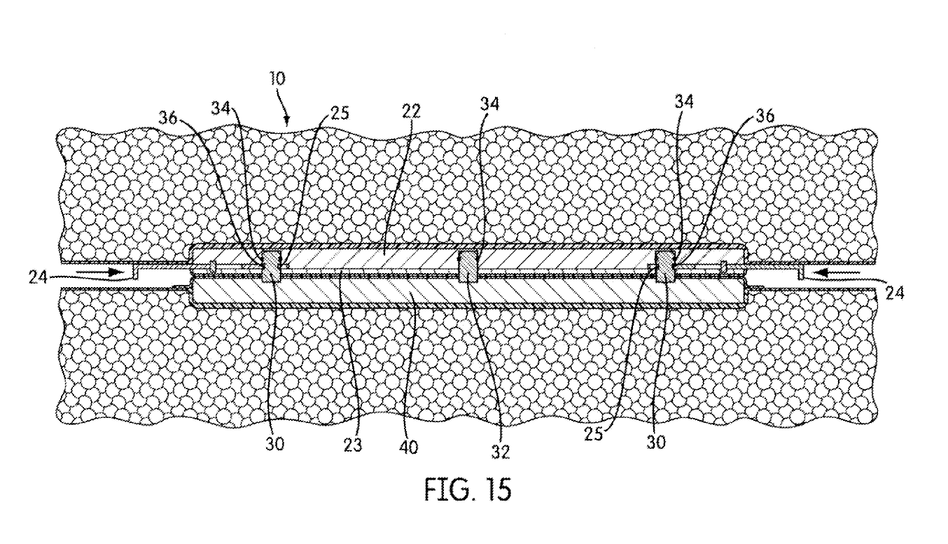

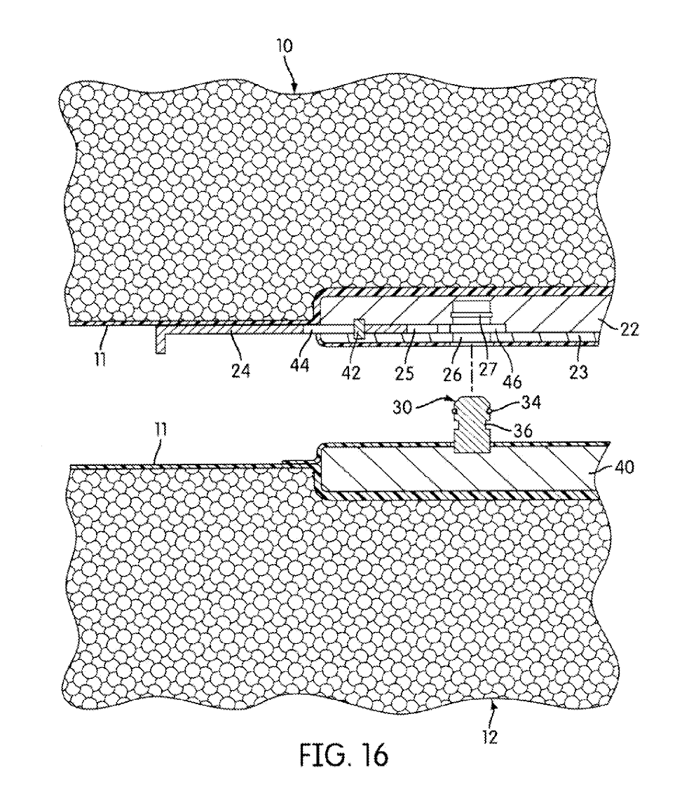

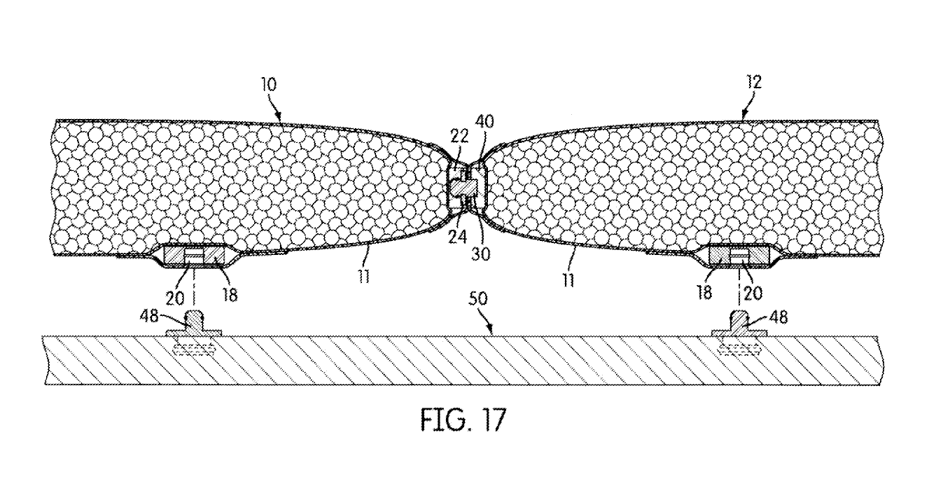

As best seen in FIGS. 11 and 12 the slide latch 24 fits into a slot 46 cut in the frame member 22. The slide latch 24 has an elongated opening 44 allowing the dowel pin 42 to pass through. When the slide latch 24 is pulled outward in the unlocked position, as shown in FIGS. 11 and 14 the dowel pin 42 prevents the slide latch 24 from extending beyond the slot 46. As shown in FIGS. 12 and 15 the slide latch is pressed inward to the locked position. The C-shaped tabs 25 of latch 24 engage the pins 30 protruding through a hole 26 of frame member 22. The thickness of the slide latch 24 allows the C-shaped tab 25 to fit in the groove 36 on the pin 30. Furthermore, as illustrated in FIGS. 15 and 16, the outer pins 30 and a center pin 32 have an O-ring 34 which correspond to a groove 27 in the outer holes 26 and center hole 28 when the upper cushion 10 and lower cushion 12 are attached.

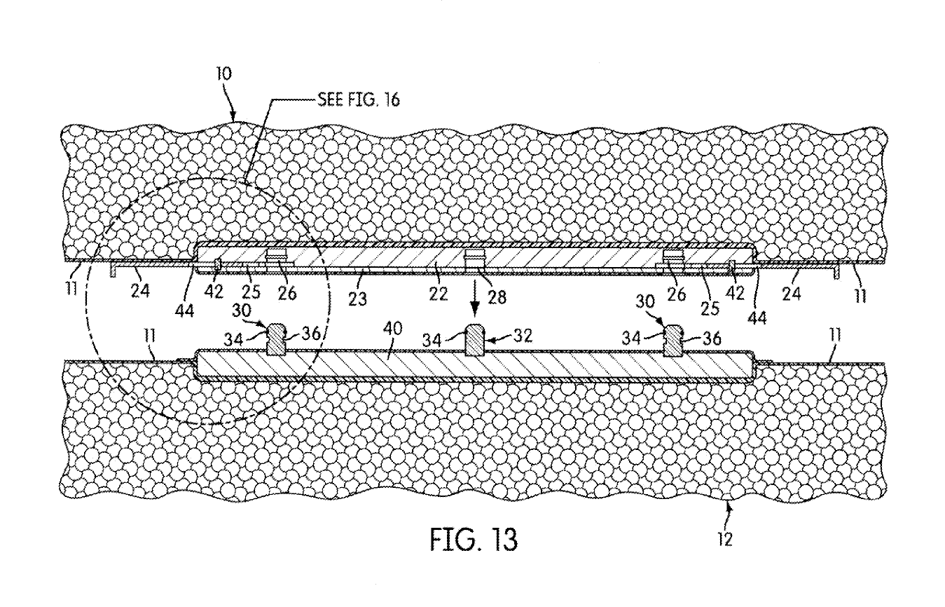

As best seen in FIGS. 13 and 14 the upper body cushion's 10 interlocking frame member 22 and lower body cushion's 12 interlocking frame member 40 are sandwiched between two layers of the outer casing 11. The outer casing 11 secures the frame members tightly in place. The interlocking frame members 22, 40 and associated components may be constructed of rigid, high strength plastics or non-ferromagnetic materials to maintain MRI compatibility. Additionally the interlocking frame members 22, 40 may be constructed using injection molding techniques.

The body cushions are initially molded around the patient at the time of simulation. Before a patient can begin treatment they first undergo imaging commonly using CT, PET-CT, MRI or a combination of these modalities. At this time the patient is positioned/immobilized in the desired treatment position. The point of attachment of the index bars to the support surface is also noted to provide the body cushions are indexed to the treatment table in the proper place. During this procedure the body cushions are molded around the patient and evacuated of air. In creating the initial mold, the patient lies on top of the attached upper body and lower body cushion with their interlocking frame members interlocked. Using CT simulation the physician is able to place an isocenter which is marked on the patient and or cushions using a three point system. It is imperative that the patient's position and immobilization are reproducible for treatment or additional imaging procedures.

The following paragraph describes a preferred embodiment of a method of use for the use of the interlocked vacuum body cushions on a treatment table. The upper body cushion 10 is attached to the treatment table using index bars 48. The round ends of the index bars are fixed to the treatment table 50 using the semi-circle indents along the longitudinal edge of the surface. The pins of the index bar 48 project upward to meet the holes 20 of The cushion position frame members 18. The upper body cushion 10 is now securely fixed to the treatment table 50. In another possible embodiment, the body cushions may be placed on top of some other positioning or immobilization device which is indexed to the treatment table. The patient is then able to seat themselves on the treatment table 50 just below the end of the upper body cushion 10 containing the interlocking frame member 22. The patient is then able to move their body backwards until their buttocks are in the upper body cushion. At this point the patient may lay back into the upper body cushion 10. The patient's legs can now be elevated and the lower body cushion 12 placed under his/her legs. The lower body cushion 12 is mated to the upper body cushion 10 using the interlocking frame members 22 and 40. The slide latches 24 are slid into the locked position FIG. 12, locking the upper body cushion 10 and the lower body cushion 12 together. The lower body cushion 12 is secured to the treatment table 50 using an index bar 48. The previous process is reversed to allow the patient to exit the cushions.

The advantages of the present invention include, without limitation, that it increases patient safety. The present device eliminates the need to have a patient overcome the height of a single molded cushion secured on top of the treatment table. Consequently, the number of clinicians needed to assist the patient is reduced. Further, repositioning tolerances are maintained by preserving the shape of the cushions. Further, the cushions shape can be adjusted independent of one another if needed without losing overall shape. For example, a patient's lower extremities experience swelling during treatment. Furthermore, at the time of simulation when the vacuum mold is created, the number of hands needed to form the cushion is reduced because each cushion can be evacuated one at a time as opposed to forming one large cushion using one valve. Finally, the separated cushions are easily transported and storage space is reduced.

Various modifications to the present invention will become apparent to those skilled in the art from the foregoing description and accompanying drawings. Accordingly, the present invention is to be limited solely by the scope of the following claims.

I claim:

1. A method of positioning, holding, and the immobilization of a patient on a radiation treatment table or patient support surface comprising the acts of:
attaching an upper vacuum cushion to said treatment table or patient support surface;
said patient seats himself/herself on said treatment table or patient supports surface just below an end of said upper vacuum cushion;
said patient moves his/her body backwards until his/her buttocks are in said upper cushion said patient lies back into back into said upper vacuum cushion;
said patient's legs are elevated and a lower vacuum cushion is placed under his/her legs;
said lower vacuum cushion is mated to said upper vacuum cushion using an interlocking frame member on said upper vacuum cushion and an interlocking frame member on said lower vacuum cushion;
said patient places lower body portion onto said lower vacuum cushion;
slide latches within said interlocking frame members are slid into a locked position, locking said upper vacuum cushion and said lower vacuum cushion together;
said interlocked cushions are secured to said treatment table or patient support surface by one or more cushion position frame members.

2. The method of claim 1 comprising the acts of:
one or more pins of an index bar projects upward to meet said one or more holes of said cushion position frame members.

3. A device for positioning, holding, and the immobilization of a patient on a patient support device comprising:
an upper vacuum cushion and a lower vacuum cushion;
a first interlocking frame member located at a proximal end of said upper vacuum cushion;
a second interlocking frame member located at a proximal end of said lower vacuum cushion;
means for interlocking said interlocking frame members;
a means for positioning said interlocked cushions to said treatment table or patient support device.

4. The device of claim 3 further comprising:
said vacuum cushions have an outer casing made from a flexible gas impermeable material;

5. The device of claim 3 further comprising:
said vacuum cushions are filled with a yieldable substance;

6. The device of claim 3 further comprising:
said vacuum cushions have a valve to inflate and deflate said cushion.

7. The device of claim 3 further comprising:
said vacuum cushions have a support ring for cushion storage.

8. The device of claim 3 comprising:
said first interlocking frame member further comprising;
one or more holes; and
one or more slide latches; or one or more pins.

9. The device of claim 8 comprising:
one or more outer pins; and
a center pin.

10. The device of claim 9 comprising:
said outer pins each have a groove and rubber O-ring; and
each center pin has a rubber O-ring.

11. The device of claim 9 comprising:
one or more outer pins accept corresponding outer holes;
a center hole accepts a corresponding center hole; and
said corresponding holes provided by mating second interlocking frame member.

12. The device of claim 8 comprising:
one or more outer holes; and
a center hole.

13. The device of claim 12 comprising:
one or more outer holes that accept corresponding outer pins;
a center hole accepts a corresponding center pin; and
said corresponding holes provided by mating first interlocking frame member.

14. The device of claim 3 comprising:
said second interlocking frame member further comprising;
one or more holes; and
one or more slide latches; or one or more pins.

15. The device of claim 14 comprising:
one or more outer pins; and
a center pin.

16. The device of claim 9 comprising:
said outer pins each have a groove and rubber O-ring; and
each center pin has a rubber O-ring.

17. The device of claim 14 comprising:
one or more outer holes accept corresponding outer pins;
a center hole accepts a corresponding center pin; and
said corresponding pins provided by mating first interlocking frame member.

18. The device of claim 14 comprising:
one or more outer holes; and
a center hole.

19. The device of claim 11 comprising:
one or more outer holes that accept corresponding outer pins;
a center hole accepts a corresponding center pin; and
said corresponding pins provided by mating first interlocking frame member.

20. A method of positioning, holding, and the immobilization of a patient on a radiation treatment table or patient support surface comprising the acts of:
using interlocked vacuum cushions on a treatment table further comprising the acts of.
attaching an upper cushion to said treatment table using one or more index bars, one or more round ends of said index bars fixed to said treatment table using one or more semi-circle indents along a longitudinal edge of said surface;
one or more pins said index bar projects upward to meet one or more holes of said frame members;
with said upper cushion in place, said patient seats himself/herself on said treatment table just below an end of said upper cushion containing an interlocking frame member;
said patient moves his/her body backwards until his/her buttocks are in said upper cushion said patient lies back into back into said upper cushion 10;
said patient's legs are elevated and a lower cushion is placed under his/her legs;
said lower cushion is mated to said upper cushion using said interlocking frame members from said upper cushion and an interlocking frame member from said lower cushion;
slide latches within said interlocking frame members24 are slid into a locked position, locking said upper cushion and said lower cushion together;
said interlocked cushions are secured to said treatment table using a lower cushion index bar.

21. A device for positioning, holding, and the immobilization of a patient on a radiation treatment table or patient support surface comprising:
an upper cushion and a lower cushion;
a first interlocking frame located at a proximal end of said upper cushion;
said first interlocking frame member further comprising one or more holes and one or more slide latches;
said outer holes accept corresponding outer pins and center hole accepts a corresponding center pin;
a second interlocking frame located at a proximal end of said lower cushion;
said interlocking frame member further comprising one or more holes and/or one or more slide latches;
said outer holes accept corresponding outer pins and center hole accepts a corresponding center pin;
said outer pins each have a groove and rubber O-ring; and
each center pin has a rubber O-ring.
